(19) United States
(12) Patent Application Publication
(10) Pub. No.: US 2022/0117537 A1
(43) Pub. Date: Apr. 21, 2022

(54) COMMON MODE REJECTION CONFIGURATION FOR IMPROVING SPATIAL RESOLUTION

(71) Applicant: CoreMap, Inc., Colchester, VT (US)

(72) Inventor: Peter S. Spector, Colchester, VT (US)

(21) Appl. No.: 17/507,102

(22) Filed: Oct. 21, 2021

Related U.S. Application Data
(60) Provisional application No. 63/094,735, filed on Oct. 21, 2020.

(51) Int. Cl. A61B 5/305; A61B 5/287; A61B 5/367

(52) U.S. Cl. CPC A61B 5/305; A61B 5/367; A61B 5/287

(57) ABSTRACT

The present disclosure describes a common mode rejection (CMR) electrode configuration. A CMR electrode configuration improves the spatial resolution of electrogram recordings by increasing the size of a region of cardiac tissue that contributes to the electrogram recording.

### FIELD OF INVENTION

This disclosure applies to electro-anatomical mapping. Such mapping can map persistent atrial fibrillation for the purposes of guidance for ablation therapy.

### BACKGROUND

Electro-anatomical mapping of the heart is a technique commonly used in cardiac electrophysiology to plan, optimize, and verify ablation therapy for a wide variety of cardiac arrhythmias. In typical use, an intra-cardiac mapping catheter with one or more electrodes collects electrical data from the endocardial (or epicardial) surface to determine local activation timing for multiple locations on the surface of the heart chamber. By taking advantage of similarity between beats, data from multiple beats may be combined to form a map of activation across an entire chamber, thereby revealing candidate ablation target sites, which have the highest likelihood of terminating and preventing arrhythmia while sparing healthy cardiac tissue.

However, unlike periodic arrhythmias, such as flutter, fibrillation is not amenable to the same type of multi-beat activation mapping described above, as there is no timing reference common to all beats, which is required for stitching together “beats”. Furthermore, due to the complexity of electrical differences between heart cells that occur over smaller distances, it is common to obtain complex fractionated signals.

Current technologies cannot effectively ascertain patient-specific maps of fibrillation. Thus, treatments are limited to “one size fits all approaches”. Unfortunately, antiarrhythmic medications are only effective in approximately half of the 33 million patients suffering from, for example, atrial fibrillation (AF). Accurate activation mapping is further complicated by the complexity of projecting sequentially acquired local activation time measurements from a region in 3D space, onto a stationary representation of the heart's surface. Challenges due to this projection can result in erroneous interpretation of conduction direction.

Catheter ablation has emerged as the treatment of choice for patients experiencing drug-resistant AF. (Calkins et al., 2(4) Circ. Arrhythmia Electrophysiol., 349-61 (2009)). Unfortunately, current ablation methods for AF fail for approximately 30% of patients. Id. at 354. Despite the difficulties in AF patients, ablation when used in patients with other heart arrhythmias achieves a 95% success rate. (Spector et al., 104(5) Am. J. Cardiol., 671, 674 (2009)).

The relative lack of success for AF patients arises largely from the inability of prior technologies and methods to accurately map and determine the source locations and mechanisms leading to AF in individual patients. AF is characterized by complex, variable self-perpetuating electrical activities in the heart. This presents a two-fold problem. First, the source locations and mechanisms leading to AF differ between patients. Second, the complex and variable nature of source locations and mechanisms leading to AF make them difficult to map and determine. Consequently, physicians are left with treating patients using generalized strategies that fail to account for the unique presentation of AF in individual patients. In terms of ablation, this often means that sources of AF are left untreated. Concurrently, healthy heart tissue is ablated, which can actually increase a patient's likelihood of developing arrhythmias.

### BRIEF DESCRIPTION OF THE DRAWINGS

FIG. 1: an exemplary CMR electrode configuration measuring a propagating cardiac activation signal.

FIG. 2: unipolar, bipolar, and CMR electrode measurements using the CMR electrode array of FIG. 1.

### DETAILED DESCRIPTION

The present disclosure describes a common mode rejection (CMR) electrode configuration. A CMR electrode configuration improves the spatial resolution of electrogram recordings by increasing the size of a region of cardiac tissue that contributes to the electrogram recording. This electrode design, and methods, systems, and devices employing the design, find particular use in assessing fibrillation.

The CMR electrode configuration uses a two-dimensional array of electrodes, which may be microelectrodes, distributed across the array at known locations and each electrode is separated by a known distance. The array can be used to construct a map of cardiac rhythm and tissue properties, such as scarring. Electrodes in the array detect at least one local activation signal as a cardiac activation wave propagates through tissue underneath the array. A “central” electrode in the array that detects the local activation signal works in conjunction with multiple “surrounding” contact electrodes that surround the central electrode on the array. As a local activation signal passes underneath the central electrode a signal is concurrently recorded from both the central electrode and the surrounding electrodes. The signals from the surrounding electrodes are averaged, and the resulting average is subtracted from the central electrode signal.

As a result, the CMR electrode array is able to accurately measure the activation signal by eliminating both far-field signal interference and near-field signal interreference from other electrodes on the array. As a result, signal detected by the central electrode represents only the signal generated by the activation signal as it passes underneath the electrode. The CMR electrode array is thus able to leverage the benefits of unipolar electrodes and bipolar electrodes, while eliminating their drawbacks.

Unipolar electrodes are susceptible to both near- and far-field signal interreference. Unipolar electrodes detect a portion of the electric field generated by tissue immediately beneath the electrodes and a portion generated by tissue some distance away from the electrode site, i.e., a far-field signal. In a bipolar electrode configuration, the difference between the signal recorded at two different electrodes is measured. This has been shown to improve spatial resolution, i.e., the tissue region recorded by the electrogram. This improvement occurs because both electrodes detect the remote tissue similarly, and this far-field signal is cancelled by the bipolar recording. The difference signal results only from tissue that is close to either of the electrodes but remote from the other electrode. However, there is a disadvantage inherent in bipolar electrode recordings if both electrodes are in contact with the same tissue (a “contact bipole”)—the spatial resolution (recording region) includes the footprint beneath each electrode.

The CMR electrode array of the present disclosure improves upon both unipolar and bipolar electrode configurations.

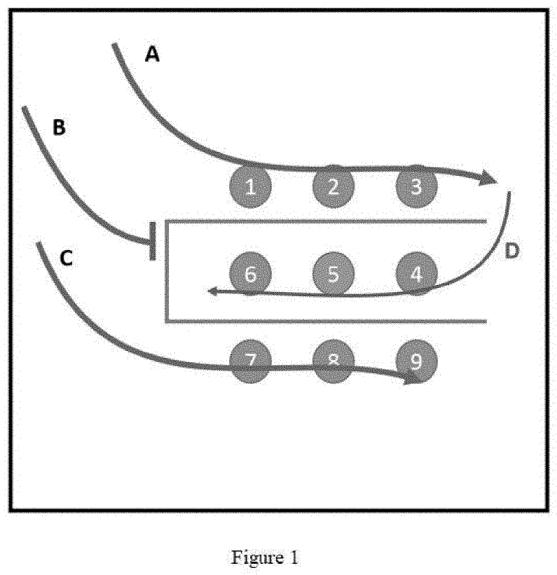

FIG. 1 illustrates an exemplary CMR electrode array of the present disclosure. The wave of a local activation signal (A, B, and C) travels from the top left to the bottom right of the figure across a tissue on which the array is placed. When the wave encounters, for example, an open-ended scar (shown as a grey three-sided rectangular shape), which is a common feature in arrythmias, the wave is cannot propagate straight through the scar. Such scars are known to not conduct excitation energy. Thus, the wave must propagate around the scar. As shown in FIG. 1, the wave (D) propagates around the scar and into the region that the open-ended scar surrounds.

As shown in FIG. 1, the electrodes of the array are positioned both within and outside the scar region. The array of electrodes comprises a series of nine electrodes, labeled 1-9, and arranged in three rows. However, other numbers and arrangements of electrodes are contemplated by the invention, for example, concentric circles, spirals, etc. The central electrode, electrode 5, is colored orange. The surrounding electrodes, electrodes 1-4 and 6-9, are colored blue.

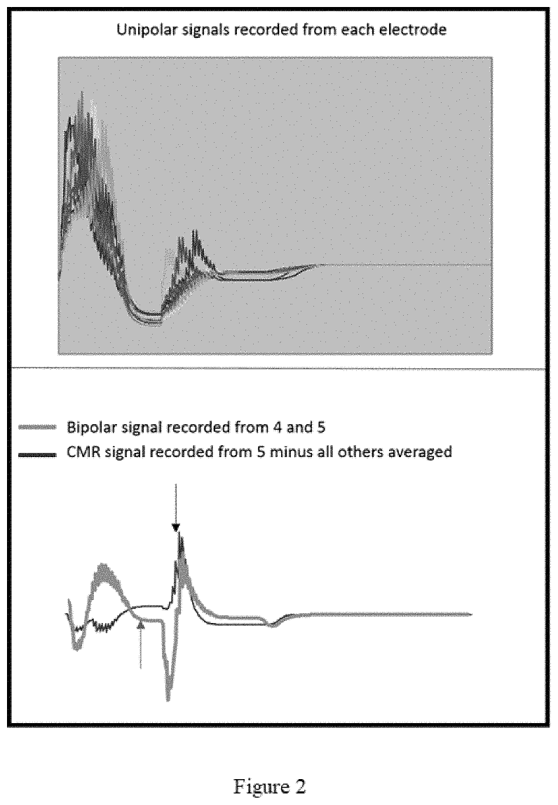

FIG. 2 shows unipolar, bipolar, and CMR electrode signal measurements using the array shown in FIG. 1. The top panel of FIG. 2 shows the unipolar measurements from each electrode in the array as the wave passes underneath electrode 5. The signals suffer from both near- and far-field signal interference. In the bottom panel the light blue line is a bipolar signal from electrode 5 minus the signal from electrode 4. Although the bipolar measurement eliminates far-field effects from outside the scar, electrode 5 nevertheless detects the wave as it passes beneath both electrode 4 (red arrow) and electrode 5 (black arrow).

The dark line in the bottom panel of FIG. 2 is a CMR signal for electrode 5, i.e., the signal measured by electrode 5 minus the average signal of the surrounding electrodes (electrodes 1-4 and 6-9). Thus, because 8 surrounding electrodes are used, the contribution of the local signal beneath each of the surrounding electrodes is diminished 8-fold relative to the contribution of the tissue beneath the central electrode. As a result, and as shown in FIG. 2, the CMR signal concurrently minimizes far-field signal from outside the scar, and detects the wave (as shown by the large deflection in the dark line) only as it passes underneath electrode 5.

The use of simultaneously obtained electrode data according to the invention enables one to determine relative positions of measurements made by multiple electrodes in the construction of a map of cardiac rhythm. Methods of the invention by which direction of activation is projected to the heart surface are unaffected by motion (e.g., cardiac or respiratory). This, then, allows the generation of a more precise cardiac map and avoids the impact that motion has on projections of non-simultaneously acquired electrode data onto the cardiac surface. In such methods, as the activation signal propagates in the tissue underneath the array, the signal is measured by a series of consecutive central electrodes. Thus, as the wave propagates, an electrode that may have been a surrounding electrode is tasked as a “new” central electrode. Concurrently, appropriate electrodes are tasked as “new” surrounding electrodes for the new central electrode.

Cardiac substrate abnormalities may manifest as variations in conduction velocity, minimum cycle length, and/or other measurable properties derived from intracardiac signals. Therefore, analysis of signals may be applied in order to deduce substrate characteristics. The CMR electrode configuration provided by the present disclosure may be used to acquire high spatial resolution signal to create a local activation map for the purpose of deriving local tissue properties.

The CMR electrode configuration may be employed on a novel catheter, on which the array of electrodes is used to resolve electrograms at high spatial resolution. By combining these electrodes, which may be micro-scale, into an array, temporal and spatial relationships of adjacent activations may be analyzed and used to create maps of distinct waves of activation. From these maps of fibrillation, the tissue properties that determine the type and distribution of arrhythmogenic drivers may be deduced. In such methods, the electrode considered the center electrode may shift as the wave propagates across the array. Consequently, the electrodes considered to be the surrounding electrodes would also shift in conjunction with the “new” central electrode.

The systems and methods of the disclosure may incorporate or use one or more catheters to which the disclosed electrode arrays are attached. The distal ends of these catheters are inserted into a patient's heart, and the electrode arrays are deployed.

Catheters of the present disclosure may also be used in conjunction with surgical devices for accessing a patient's heart, i.e., sheaths with valves, and one or more guidewires for positioning catheters.

Catheters of the disclosure may also be used in conjunction with an imaging subsystem. This can allow, for example, viewing tissue and/or the catheter while deployed inside a patient.

Catheters may also be deployed in conjunction with electrode localization technologies, including radio frequency-based localization, triangulation-based localization, and/or impedance-based localization.

The present disclosure provides systems and methods using electrode arrays that can be positioned within a patient's heart. An electrode is an electrical conductor. Electrodes of the present disclosure include electrodes, which may be solid conductors, such as needles or discs.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

What is claimed is:

1. A method for measuring a cardiac activation signal in a patient, the method comprising:
positioning a two-dimensional electrode array at a location in a patient's heart, wherein the two-dimensional electrode array comprises a central electrode surrounded by surrounding electrodes of arranged and distributed across the array;
simultaneously detecting at least one local activation signal at the central electrode and each of the surrounding electrodes; and
averaging the activation signal detected by each of the surrounding electrodes;
subtracting the average activation signal of the surrounding electrodes from the activation signal detected by the central electrode.

2. The method of claim 1, further comprising determining an activation time of the local activation signal for the activation signal.

3. The method of claim 2, further comprising calculating a velocity vector of the local activation signal.

4. The method of claim 3, further comprising compiling an isochronal activation map comprising the two-dimensional electrode array.

5. The method of claim 4, further comprising mapping the trajectory of a cardiac activation wave using the electrodes of the two-dimensional electrode array.

6. The method of claim 5, further comprising detecting a conduction block using the two-dimensional electrode array.

7. The method of claim 6, wherein the step of detecting a conduction block comprises determining that the activation times between two or more adjacent electrodes are below a threshold indicative of direct propagation of the cardiac activation wave between the two or more adjacent electrodes.

8. The method of claim 7, wherein said threshold is adjusted based on a direction of a propagation vector with respect to a putative site of conduction block.

9. The method of claim 4, further comprising calculating the spatial context for each local activation signal of each electrode of the two-dimensional array.

10. The method of claim 1, further comprising constructing a map of cardiac electrical activity using the two-dimensional array.
